(19) United States
(12) Patent Application Publication
(10) Pub. No.: US 2009/0149918 A1
(43) Pub. Date: Jun. 11, 2009

(54) IMPLANTABLE ANTENNA

(76) Inventors: Peter Krulevitch, Pleasanton, CA (US); Michael R. Tracey, Branchburg, NJ (US); Stuart Wenzel, San Carlos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/951,376

(22) Filed: Dec. 6, 2007

(51) Int. Cl. A61N 1/00

(52) U.S. Cl. 607/60

(57) ABSTRACT

An antenna implantable through minimally invasive techniques, preferably comprising a coil with conductive probes is provided. The antenna is preferably superelastic nickel-titanium having an insulative coating. The antenna may conduct a signal originating from a device external to the body of the implantee, or from another implanted device connected to the antenna depending on whether the antenna is employed for sending, receiving, or transceiving signals. Signals may contain data, operational commands, and may be used to transfer power. The implantable antenna may be connected to another implanted device, such as a blood pressure monitor, or may be implanted as a stand-alone device for purposes such as stimulating tissue.

### BACKGROUND OF THE INVENTION

1. Field of the Invention

The present invention relates to an antenna, and more particularly to an implantable device that functions as an antenna. In addition, the present invention relates to an antenna that may function as a receiver, as a transmitter, or as a transceiver for wireless data signals and power transfer. The present invention also relates to an antenna that may be implanted via a catheter below the outer surface of the skin of a patient. The present invention further relates to an antenna that includes one or more conductive probes, which may pass from the antenna to deeper portions of the patient's tissue or more remote portions of the patient's anatomy. Moreover, the conductive probes may be connected to another device implanted within the body of the patient.

2. Discussion of the Related Art

Implantable devices are well-known in the art. As the use and development of implantable devices has become more established, the possibility of diagnosing and treating medical conditions through the use of active implants has made it desirable to design devices that are able to operate for prolonged periods within the patient. For obvious reasons, the patient's quality of life will benefit from devices that are as unobtrusive as practically possible. However, as the sophistication and complexity of implantable devices increases, there may be tradeoffs between their obtrusiveness and functional capability. Size, operational capability, battery life, tissue trauma, and durability are some examples of the considerations that may conflict with the goal of preserving the minimally obtrusive nature of an implantable device design. For patients having implants used in the treatment of chronic medical conditions, the longevity of the implant may be of significant importance. Additionally, some implants need to be positioned deep within the patient (due to patient obesity or the need to treat deep tissue, for example) while maintaining communication to remote devices, such as a wireless transceiver external to the body. These deep implants present additional challenges associated with electrical signal transmission and quality, as well as invasiveness.

Patients who are implanted with devices that require interaction with devices external to the patient's body are often faced with the risk of infection at the location where the means of interaction, such as wires, physically passes through the outer surface of the patient's skin. Quality of life may be negatively impacted due to reduced mobility for the period when an implant is required to be connected to a device external to the patient. Additionally, implants capable of wireless communication may require invasive surgical procedures further potentially increasing the risk of complications such as infection while reducing the patient's quality of live through the invasive nature of the implant and implant procedure. Accordingly, there is a need to minimize an implant's intrusion into the patient's quality of life while maximizing the implant's operational performance and longevity. The use of an implantable antenna beneath the outer surface of a patient's skin is a means for addressing this need.

### SUMMARY OF THE INVENTION

The present invention overcomes the disadvantages associated with current implant designs as briefly described above.

In accordance with one aspect, the present invention is directed to an implantable inductive device. The implantable inductive device comprises an antenna having a coil section and a lead section. The coil section including one or more windings and the lead section including at least one lead positioned a predetermined distance from the coil section. At least one of the one or more windings of the coil section and at least one lead of the lead section is formed from a superelastic material. The antenna is configurable for implantation via minimally invasive medical instruments.

An implantable antenna may function as a means for transmitting, or as a means for receiving, or as a means for transceiving. The antenna is located beneath the outer surface of the patient's skin and may communicate with other devices internal or external to the patient, or the antenna may function as a stand-alone device.

In accordance with one exemplary embodiment, the present invention is directed toward functioning as a transmitter providing a means for communication from an implanted device to other devices external to the patient's body. The antenna is implanted below the outer surface of the patient's skin in a location that facilitates the wireless transmission of a signal from the patient to an apparatus external to the patient. The implanted antenna may be connected to another implanted device such as those that are capable of gathering physiological or biological information, including the patient's blood pressure. The antenna and its proximity to the body surface increases the transmission quality of the signal sent to the external devices by reducing the amount of tissue that the signal passes through before leaving the patient's body, thereby increasing the broadcast signal range while preferably reducing implant power consumption for benefits such as prolonged battery life.

In accordance with another exemplary embodiment, the present invention is directed toward functioning as a receiver providing a means for communication or power transfer to an implanted device from an apparatus external to the patient's body. The antenna is implanted below the outer surface of the patient's skin in a location that facilitates the wireless transmission of a signal to the patient from devices external to the patient. The implanted antenna may be connected to another implanted device such as those that are capable of impacting physiological or biological functions. The antenna's proximity to the body surface increases the transmission quality of the signal sent to the implanted apparatus by reducing the amount of tissue that the signal passes through, thereby increasing the broadcast signal range, received signal strength, and signal quality at the location of the implant within the body of the patient. In this exemplary embodiment, signals external to the body of the patient may be from devices compatible for use with the implant. Signals from external devices may be generated under a broad range of scenarios such as supervised medical care, on-demand by the patient, by a topically applied patch, automatically by programmed devices, and the like. Signals received by the antenna may be dedicated to power transfer, to operational command transmission, or may be combined to do both.

In accordance with yet another exemplary embodiment, the present invention is directed toward functioning as a stand-alone receiver providing a means for conducting a signal to a target site within the patient's body. The antenna is implanted below the outer surface of the patient's skin in a location that facilitates the wireless transmission of a signal to the patient from devices external to the patient. The implanted antenna may receive a signal from a device such as those that are capable of impacting physiological or biological functions. The antenna and proximity to the body surface increases the efficacy of the signal sent to the target site within the patient's body by reducing the amount of tissue that the signal passes through. The antenna may be insulated in such a manner as to prevent untargeted tissue adjacent to the antenna from conducting the signal delivered by the antenna. For example, the antenna may be implanted for the purpose of stimulating a target nerve located near other nerves at some distance from the surface of the skin. The antenna allows the target nerve to receive the signal while preferably avoiding significant signal degradation due to current leakage into adjacent tissue. Additionally, the use of an insulated antenna and probes preferably avoids the signal being received by other untargeted nerve tissue. Signals from external apparatuses may be generated under a broad range of scenarios such as supervised medical care, on-demand by the patient, by a topically applied patch, automatically by programmed devices, and the like.

In accordance with yet another exemplary embodiment, the present invention is directed toward functioning as a transceiver providing a means for passing signals from an implant, within the body of the patient, to devices external to the body of the patient, and vice-versa. A transceiving antenna may be used for purposes such as real-time monitoring and response to a patient's life functions including the patient's blood pressure, power transfer from devices external to the patient, or both. For example, a coil antenna may be used to transmit data from an implant located more remotely from the antenna, within the body of the patient, to an apparatus located external to the patient. The apparatus may then analyze the transmitted data and provide an operational command to the remotely located implant via the coil antenna, which is positioned under the outer surface of the patient's skin. In this example, an operational command may be automatically generated, may be partially automated and partially manually generated, or may entirely manually generated. For instance, a signal may be generated under automated feedback control based on data from the implant that may be processed internally or externally to the body.

In each of the exemplary embodiments described above, the antenna may comprise any biocompatible material or materials suitable for conducting and transmitting a signal. The coil antenna may be flexible. More generally, the implanted system may comprise flexible and non-flexible parts. For example, the implant may have a small-diameter, rigid coil antenna under the skin surface that is connected via flexible leads to remote tissue, or to a remote device such as a pressure sensor. One preferable material is a shape-memory/superelastic alloy such as a nickel-titanium alloy, which easily lends itself to implantation using common devices such as catheters. Moreover, it is preferable that the material of composition be capable of tolerating the range of motion that a patient may require for limb movement. The insulating material may comprise of any biocompatible material that preferably adheres to the outer surface of the antenna in a manner that allows the antenna to remain flexible. One preferable example of insulating material is Parylene. Another example is silicon carbide (SiC), which may be deposited by chemical-vapor deposition techniques or any other suitable techniques.

In each of the exemplary embodiments described above, the antenna is preferably in the form of a coil, but further optimizations in form may be made for purposes such as enhanced tissue ingrowth, signal strength, mechanical flexibility, and the like. Such optimizations may include antenna forms other than a coil, antenna forms in combination with a coil, and antenna forms combined with other forms such as a mesh. The antenna may also possess more than one probe passing from a location near the surface of the patient's skin to tissue farther below, or more remote from the location of the antenna.

### BRIEF DESCRIPTION OF THE DRAWINGS

The foregoing and other aspects of the present invention will best be appreciated with reference to the detailed description of the invention in conjunction with the accompanying drawings, wherein:

FIG. 1 is a schematic view of a first exemplary coil antenna terminating in a single conductive probe wherein the antenna and probe are coated with an insulating layer in accordance with the present invention.

FIG. 1A is a schematic view of a second exemplary coil antenna terminating in a single conductive probe wherein the antenna and probe are coated with an insulating layer in accordance with the present invention.

FIG. 2 is a schematic view of an alternate exemplary embodiment of the device illustrated in FIG. 1, wherein the coil antenna terminates with two conductive probes in accordance with the present invention.

FIG. 3 is a schematic view of the coil antenna illustrated in FIG. 2, wherein the antenna is attached to an implanted device used to monitor pressure in accordance with the present invention.

### DETAILED DESCRIPTION OF THE PREFERRED EMBODIMENTS

An implantable antenna may function as a means for transmitting, or as a means for receiving, or as a means for transceiving. The antenna is located beneath the outer surface of the patient's skin and may communicate with other devices internal or external to the patient, or the antenna may function as a stand-alone device.

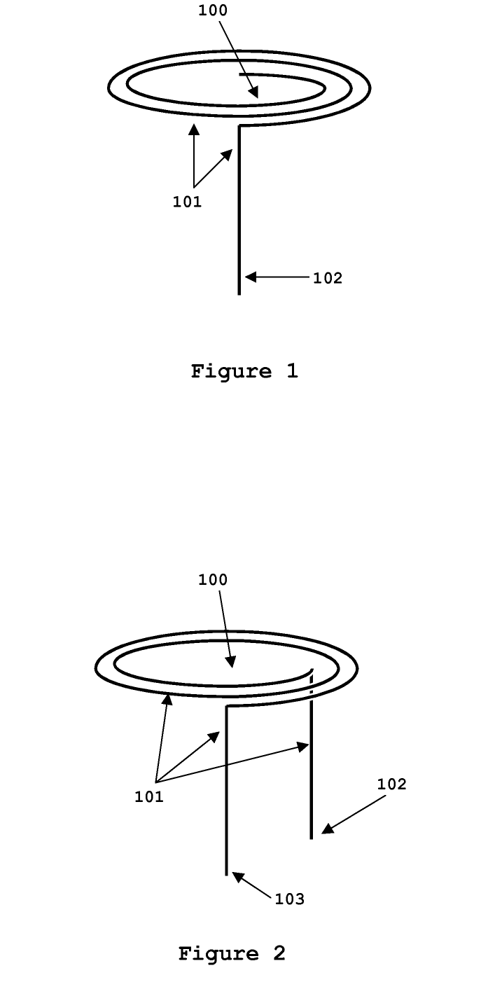

FIG. 1 illustrates a preferred exemplary embodiment wherein the antenna comprises a coil 100 and a conductive probe 102. The coil 100 and the probe 102 are preferably coated with an insulating layer 101. The number of turns in coil 100 ranges from about 1 to about 10,000. The end of the antenna not comprising the coil 100 preferably terminates in the conductive probe section 102 of the antenna. The coil 100 and conductive probe 102 are preferably made from a continuous piece of biocompatible conductive material. A preferred exemplary embodiment of the antenna's coil 100 and probe 102 is made from a superelastic alloy such as nickel-titanium comprising from about 50.0 weight percent nickel to about 60 weight percent nickel, with the remainder being titanium. Preferably, the antenna's coil 100 and probe 102 are designed such that they are superelastic at body temperature, having an austenitic finish temperature of about twenty-four degrees Celsius to about thirty-seven degrees Celsius. The antenna's material of construction preferably allows implantation using a minimally invasive technique such as catheter-based delivery or injection.

The insulating layer 101 illustrated in FIG. 1, preferably covers the surfaces of coil 100 and probe 102 to enhance the operational efficiency of the antenna by avoiding the conduction of the signal away from the antenna and into the patient's surrounding tissue. The insulating layer 101 may comprise any available material that is biocompatible, preferably preserving the range of motion of the coil 100 and probe 102 that a patient may desire for limb movement. One preferable example of insulating material is Parylene. Another example is silicon carbide (SiC). Either of these materials may be deposited by chemical-vapor deposition techniques or any other suitable technique known in the relevant art.

The exemplary embodiment illustrated in FIG. 1 may function in a number of ways. First, the antenna may be connected via the probe 102 to another implanted device, wherein this device sends a signal to yet another device external to the body of the patient via the probe 102 and coil 100. Most preferably, the coil 100 is located below the outer surface of the patient's skin. Second, the exemplary embodiment of FIG. 1 may be connected via the probe 102 to another implanted device, wherein this device sends and receives (transceives) signals with yet another device external to the body of the patient via the probe 102 and coil 100. Third, the exemplary embodiment illustrated in FIG. 1 may be implanted to function as a stand-alone device wherein coil 100 receives a stimulating signal, which is preferably conducted to a discrete location within the anatomy of the patient via the probe 102 for purposes such as nerve stimulation. Optionally, the insulating layer 101 may be included for optimizing the conduction of the stimulating signal through the coil 100 and probe 102 such that the discreetly targeted location within the patient's body preferably receives the substantial portion of the stimulating signal sent through the antenna from a device external to the patient's body.

FIG. 1A illustrates a similar antenna device to that illustrated in FIG. 1 in which the relative surface areas of the electrodes (the non-insulated ends of the conductor at the antenna coil 102b and at the probe tip 102a) are changed relative to one another, in order to change and improve electrical efficiency. In operating as a receiver, coil antennas such as these convert an oscillating external magnetic field into a voltage V(t) between the two ends of the coil; this voltage then produces current I(t) (Amps) that depends on the resistance R between the two electrodes. In the case of direct tissue stimulation, the resistance R depends on the resistivity ρ (ohm-cm) and geometry of the tissues between the 102a and 102b. In addition, the local current density J (Amps/cm²) and electric field E (V/cm) around electrodes 102a and 102b depend on the voltage as well as the shape of the electrodes. In particular, current density is proportional to electric field through Ohm's law J=E/p. In FIG. 1A, the electrode 102b at the coil section has a much larger area than the electrode 102a at the probe tip. Since current is continuous, the same current I(t) passes through both 102a and 102b, which means that the current density J around the smaller electrode 102a must be larger than the current density around 102b. In accordance with Ohm's law above, the small electrode 102a will also have higher local electric field. Since electric field is the spatial gradient of voltage (E=−∇V), most of the voltage will be dropped in regions of high electric field, i.e., the tissue surrounding the smaller electrode. In summary, a small-area probe tip, such as 102a, may be used to concentrate the electric field, current and voltage in a desired location, such as a near to a nerve. This is advantageous because many biological effects and responses, such as nerve stimulation, depend strongly on the local electric field. The large-area electrode may be implemented in many ways, for example, it might simply be a length of circular wire or conductor that presents a larger surface area than the small-area electrode; or it might be a perforated structure (FIG. 1A) that is permeable to allow tissue growth, blood flow, or even tissue in-growth. Furthermore, the coil and material may be coated or treated with other materials to promote or retard the reaction of specific tissues. For example, Oxidized Regenerated Cellulose (ORC) fabric will minimize tissue attachment and formation of scar tissue, thus reducing the risk of coil encapsulation. Small area electrodes may be implemented using sharp tips, or with small holes in an insulator surrounding a conductor. Such hole might be made with micromachining techniques such as photolithography, laser patterning or drilling.

FIG. 2 shows an alternate exemplary embodiment of the present invention that is substantially similar to the device illustrated in FIG. 1 but with two conductive probes 102 and 103 extending from the coil 100, with the optional presence of insulating layer 101. The construction and composition of the antenna are preferably the same as that described with respect to the device illustrated in FIG. 1 with the additional feature that the termination points of the turns comprising coil 100 denote the starting points of the conductive probes 102 and 103.

The alternate exemplary embodiment shown in FIG. 2 may preferably function in the same manner as that described for FIG. 1 in that the antenna may transmit, transceive, or receive signals depending upon the antenna's intended use as described in FIG. 1. The presence of two conductive probes 102 and 103 may preferably enhance the operational efficiency of the antenna for a specific purpose. The purpose may include applying an electrical voltage and current to a very small volume, such as across the diameter of a nerve, in which case the probe tips may be located proximate to each other with the nerve in between. Alternately, the purpose may include the capability to preferably stimulate an increased area of the patient's anatomy, such as along a long nerve or across a large muscle, in which case the probe tips will be located appropriately far apart from one another.

In yet another alternate exemplary embodiment, the device of FIG. 2 may be constructed in a manner analogous to the device illustrated in FIG. 1A. In other words, a large area electrode at one termination of the antenna causes the local electric field and voltage drop to be low compared to those of the distal termination, which in this embodiment is two leads 102 and 103.

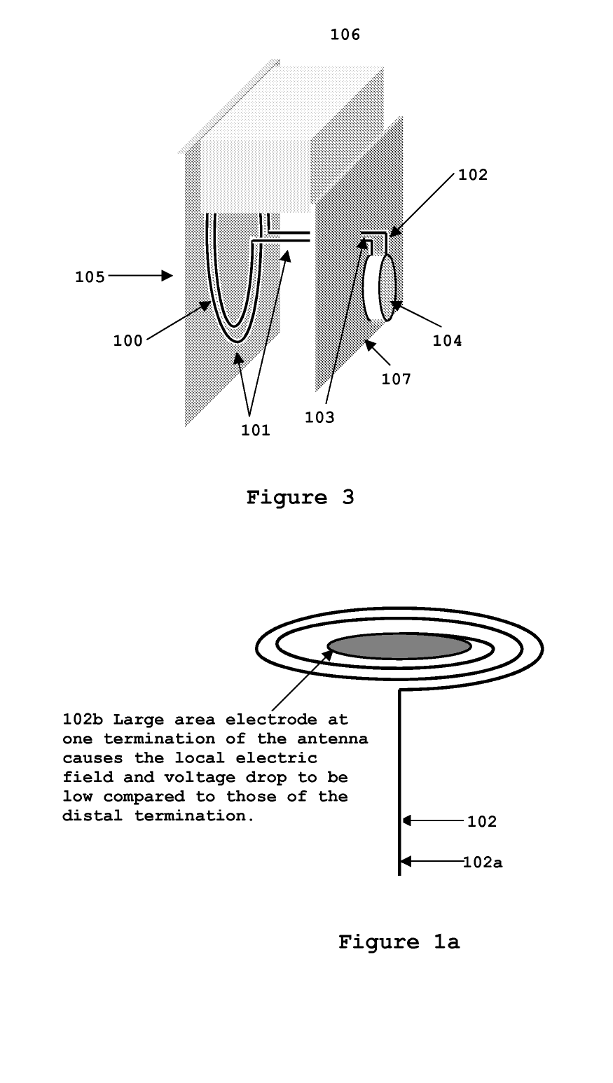

FIG. 3 illustrates the alternate exemplary embodiment of FIG. 2 attached to a blood pressure monitor 104 that is positioned within the lumen 107 of the patient's vasculature. The coil 100 is preferably located just below the outer surface of the patient's skin 105 with conductive probes 102 and 103 passing though the tissue 106 laying between the lumen 107 and the patient's outer skin surface 105. The optional insulating layer 101 is present to preferably avoid tissue 106 from conducting the signal away from the coil 100 and blood pressure monitor 104.

Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope for the appended claims.

What is claimed is:

1. An implantable inductive device comprising an antenna having a coil section, including one or more windings, and a lead section including at least one lead positioned a predetermined distance from the coil section, at least one of the one or more windings of the coil section and the at least one lead of the lead section being formed from a superelastic material, the antenna being configurable for implantation via minimally invasive medical instruments.

2. The implantable inductive device according to claim 1, wherein the superelastic material comprises a nickel-titanium alloy.

3. The implantable inductive device according to claim 2, wherein the nickel-titanium alloy comprises from about 50 weight percent to about 60 weight percent nickel and the remainder titanium.

4. The implantable inductive device according to claim 1, wherein the antenna is coated with a biocompatible, insulative coating.

5. The implantable inductive device according to claim 1, wherein the antenna is configured for communication with other devices.

6. The implantable inductive device according to claim 1, wherein the antenna is configured as a stand alone device.

7. The implantable inductive device according to claim 1, wherein the at least one lead is configured for direct stimulation of body tissue at the implant site.

8. The implantable inductive device according to claim 1, wherein the device comprises a receiver.

9. The implantable inductive device according to claim 1, wherein the devices comprises a transmitter.

10. The implantable inductive device according to claim 1, wherein the devices comprises a transceiver.
